(19) United States
(12) Patent Application Publication
(10) Pub. No.: US 2020/0146853 A1
(43) Pub. Date: May 14, 2020

(54) Intravascular Stent, Especially for Coronary Vessels

(71) Applicant: UNIWERSYTET MIKOLAJA KOPERNIKA W TORUNIU, Torun (PL)

(72) Inventors: Maciej Gagat, Bydgoszcz (PL); Alina Grzanka, Bydgoszcz (PL); Dariusz Grzanka, Bydgoszcz (PL)

(21) Appl. No.: 16/630,603

(22) PCT Filed: Jul. 12, 2018
(86) PCT No.: PCT/PL18/50035

§ 371 Date: Jan. 13, 2020

(30) Foreign Application Priority Data
Jul. 13, 2017 (PL) P.422210

(51) Int. Cl. A61F 2/89; A61L 31/10; A61L 31/16; A61F 2/915; A61L 31/14

(52) U.S. Cl. CPC A61F 2/89; A61L 31/10; A61L 31/16; A61L 2300/258; A61L 31/148; A61F 2002/91533; A61L 2300/256; A61F 2/915

(57) ABSTRACT

An intravascular stent includes cut-outs forming segmented patterns of the stent construction at the same time form the elongated lines of the main segment situated around the longitudinal stent axis and are connected via U-shaped connecting elements. This creates around the longitudinal axis of the stent a geometric pattern resembling a meander of gentle edges, and the two curves in the shape of a the letter “V” with rounded edges. The stent also includes an oval plate form a connecting segment to connect with the connecting elements of the elongated lines of the main segment, wherein every next main segment is a mirror reflection of the previous segment and in that the curves of the connecting element shaped like the letter “V” with rounded edges are a mutual mirror reflection in relation to the oval plate of the connecting segment. The stents can include a covering of various drugs.

The subject matter of the invention is an intravascular stent, especially for coronary vessels, used in medicine, especially in interventional cardiology, to be implanted at the site of a stenosis, the stent being a scaffold supporting the vessel wall and maintaining a proper lumen of the vessel.

Angioplasty techniques and stent implantation are widespread worldwide and are an alternative option to therapeutic methods, including the coronary artery bypass surgery, with a view to improving blood flow to the myocardium. The stent is implanted at the stenosis site most often using a balloon catheter, which is inserted through the femoral or radial artery. The intervention consists in expanding the balloon catheter, thus dilating the stenosis, and perpetuating the effect by implanting the stent.

Despite many successes in this field of medicine, i.e. the application of classic stents, high-pressure inflation and also the introduction of drug-eluting stents, restenosis and/or late and very late thrombosis remain major limitations of the invasive cardiology and involve re-interventions. Restenosis is most often defined by angiographic criteria relating to a stenosis at ≥50% at the site of the previous intervention. After a successful implantation of the coronary stent, the above reduction in the vessel lumen is almost entirely caused by the formation of neointima, and not by the elastic recoil or negative remodelling of the vessel wall, which both accompany the conventional balloon angioplasty surgeries without stent implantation. The formation of neointima typically lasts about 6 months following the stent implantation procedure.

In-stent thrombosis, unlike restenosis, is still one of the most serious complications following percutaneous coronary intervention involving stent implantation. Furthermore, the incidence of this complication is increased after implantation of drug-eluting stents, as compared to classic stents. Based on the time that has elapsed since stent implantation, in-stent thrombosis is classified as early (up to 30 days after stent implantation), late (≥30 days) and very late (>12 months). The incidence of late and very late in-stent thrombosis is increased in patients with implanted drug-eluting stents, but these are extremely effective in preventing the occurrence of restenosis. Concerns about this treatment method are raised by a minor but statistically significant increase in the incidence of very late thrombosis at the stent implantation site in patients treated with drug-eluting stents, which may be associated with increased heart attack mortality and morbidity as compared to persons treated with classic stents.

The application of drug-eluting stents not only reduces the formation of neointima, but also disrupts the healing process of the vessel wall. After the implantation of drug-eluting stents, a delayed and most often only partial endothelializtion of the stent surface is also observed. Hence, increased blood thrombogenicity due to increased tissue factor expression and lack of adequate thrombocyte inhibition as well as impaired blood flow caused by the remodelling of the vessel wall and endothelial dysfunction contribute to the formation of thrombus within drug-eluting stents.

The aim of the inventive stent construction is to create an effective intravascular stent solution, which will, on the one hand, significantly reduce the possibility of restenosis and, on the other hand, facilitate and accelerate the re-endothelialization of the stent implantation site and the stent inner surface.

An intravascular coronary stent in the form of a tube, as is known from Polish patent description no. 210204, is a repeated, symmetrical segmented pattern made by cut-outs on the lateral surface of a tube of a continuous material. These cut-outs form a wavy line pattern, wherein the wavy lines situated along the stent axis are the wavy lines of a parallel segment and are connected to two circumferential wavy lines of a transverse segment. The wavy lines of the transverse segment are, by contrast, connected alternately via connectors.

Also known from Polish patent description no. 210205 is an intravascular coronary stent in the form of a tube, the tube having a segmented strip pattern made by cut-outs from a continuous material, wherein the strips situated along the stent axis are connected via bends and are S-shaped strips of a parallel segment. V-shaped strips of a transverse segment are situated between the two S-shaped strips of the parallel segment, with the V-shaped strips connecting the bends of two adjacent S-shaped strips of parallel segments. In addition, the next transverse segment along the stent is arranged in the opposite direction in relation to the previous transverse segment.

From description of international publication WO2016101566A1 of patent application no. PCT/CN2015/082156 there is known an expansible cardiovascular stent of a reticular and tubular construction made of metal or a biodegradable material, the stent having a covering in the form of a membrane of collagen or collagen-chitosan composite material, or collagen-calcium phosphate composite material of suitable extensibility.

Korean patent description KR101251576 discloses an antibody coated stent, which is useful in treating blood vessel-related diseases. The invention includes a stent body, an anti-VE-cadherin antibody layer and a bio-compatible matrix arranged between the antibody layer and the stent body. The bio-compatible matrix coating the stent body is polyvinyl alcohol, polyurethane, poly-L-lactic acid, cellulose ester, polyethylene glycol, carboxy methyl dextran, collagen, fibronectin, cellulose or amorphous carbon.

From the publication of US application no. US20060013855A1 there is known a bioactive stent for type II diabetics, the stent being coated with a biodegradable and biocompatible polymer, to which is attached a ligand that specifically captures progenitors of endothelial cells. The bioligand is a peptide that specifically binds to an integrin receptor on progenitors of endothelial cells.

The existing solutions do not work in a comprehensive and targeted manner.

The aim of this invention is to increase the effectiveness of vasodilation procedures, especially dilation of coronary arteries, via balloon angioplasty with stent implantation and thus to significantly limit postoperative complications.

The essence of the construction of the inventive intravascular stent, especially for coronary vessels, is its alternate, two-segment construction made by cut-outs from a tube of continuous material, with outer main segments defining the contour of the stent edge.

It is preferred that the cut-outs of continuous material form main segments of a geometric pattern resembling a meander of gentle edges, and the number of the main segments is adapted to the length of the stent but is not less than 3.

It is also preferred that the main segments of the stent are a mirror reflection of each other and the both extreme main segments of the stent have every second outer connecting element to connect elongated lines terminating in gentle passageways with a plate in the shape of a round-point spade.

The connecting segments of the inventive stent, especially for coronary vessels, are arranged parallel to each other and are attached alternately to the next connecting element of the elongated lines of the next main segment thus forming an alpha helix pattern.

It is preferred that each connecting segment of the stent has an oval plate and the curves emanating from the plate are in the shape of a sickle or the Latin letter “V” with rounded edges and are a mutual mirror reflection in relation to the oval plate.

It is most preferred that the connecting segments of the stent are attached to every second connecting element of the elongated lines of the main segment.

Alternatively, it is possible to make the inventive intravascular stent, especially for coronary vessels, of a material of a reduced visibility in X-rays.

It is then preferred that the markers, especially of platinum or tantalum or gold, are placed within rings formed from the mentioned plate stent elements, which eliminates the necessity to place markers as additional stent elements.

The construction of the inventive intravascular stent, especially for coronary vessels, does not exclude the possibility of the stent being covered with an additional surface layer which reduces thrombogenicity.

The essence of the inventive stent solution is also the fact that the outer surface of the intravascular stent, especially for coronary vessels, is a convex structure located centrally on the outer surface of the stent construction elements and comprises drugs which inhibit cellular proliferation, especially rapamycin derivatives.

It is preferred that the outer stent covering is spaced apart from the edge of the stent elements and takes from 10% to 50% of the outer surface area of a stent construction element.

It is preferred that the outer stent covering is located centrally along the outer plane of the elements of the stent main segment.

It is most preferred that the outer stent covering is located on the outer surface of the oval plate of the connecting segment and is shaped in the shape of a “+” symbol or the letter “X”.

It is also preferred that the outer covering of the outer surface of the plate-like end of the connecting elements to connect the elongated lines of the supreme main segments is shaped in the shape of letter “Y” in such a manner that the base of the letter is a prolongation of the outer covering of the extreme main segments of the stent.

It is also essential that the covering of the inner surface of the construction of the inventive intravascular stent, especially for coronary vessels, is two-layered and includes an inner surface of the stent construction, which are monoclonal anti-CD144 antibodies being immobilised covalently or in a film.

It is most preferred that this layer covers the entire stent inner surface.

The outer layer, however, of the inner covering of the inventive intravascular stent, especially for coronary vessels, is located centrally on the inner surface of the stent construction, the inner surface being covered with monoclonal anti-CD144 antibodies, and comprises a system of cellular induction of tropomyosin-1 expression, especially covalent or electrostatic complexes of cell-penetrating peptides together with CRISPR/dCas9 system activating the tropomyosin-1 expression or with expression vectors determining the expression of human recombinant tropomyosin-1, or with stabilised mRNA molecules coding human tropomyosin-1.

It is most preferred that the outer layer of the inner stent covering is spaced apart from the edge of stent elements, taking from 50% to 90% of the inner surface area of a stent construction element.

It is preferred that the outer layer of the inner stent covering located on the inner surface of the oval plate of the connecting segment is shaped in an oval shape.

It is preferred that the outer layer of the inner covering of the inner surface of the plate-like end of the connecting elements to connect the elongated lines of the supreme stent segments is shaped in the shape of a round-point spade.

Where the stent construction is made of a material of a reduced visibility in X-rays, it is preferred that the two-layered inner stent covering also includes the surfaces of the marker-filled rings formed from plate-like stent elements.

The primary advantage of the new construction of the inventive intravascular stent, especially for coronary vessels, is its high resistance to external forces, with proper pliability being at the same time maintained. The connecting segments of the inventive stent form an alpha helix pattern and allow for an easy passage of the stent through the turns of the proximal coronary vessel sections and for the implantation at the stenosis site and the main segments maintain proper rigidity of the stent and, by supporting the blood vessel wall, maintain the dilated lumen of the blood vessel. Apart from that, each main segment is the mirror reflection of the previous main segment along the longitudinal stent axis and the connecting segments are attached alternately to the next connecting element of the elongated lines of the next main segment, which makes it possible to suitably fit the oval plates of the connecting segments between the main stent segments during stent implantation. Repetitiveness in the arrangement of individual elements provides good apposition of the stent to the vessel wall over the entire length of stent implantation, thus providing at the same time an optimal surface of the stent cut-outs for the process of re-endothelialization of the dilated vessel site. Moreover, the plate-like stent elements increase the surface area of action of the inner function coatings of the stent surface and are an additional barrier separating the outer stent covering from the inner surface of the blood vessel wall.

The new inventive intravascular stent, especially for coronary vessels, makes it possible, on the one hand, to inhibit the proliferation and migration of the cells building the vessel wall and, on the other hand, accelerates the re-endothelialization of the stent implantation site and the endothelialization of the stent surface from the bloodstream side. The convex structure of the outer coating of the new stent, by entering the blood vessel wall during stent implantation, provides desired penetration of the wall with a view to gradually releasing the drugs inhibiting cellular proliferation and the proper arrangement of the coating isolates the action of the cellular proliferation inhibiting compounds without limiting the re-endothelialization processes of the stent implantation site. The inner coating of the inventive stent interacts in turn with the late endothelial progenitor cells and with vascular endothelial cells, thus accelerating the re-endothelialization of the stent implantation site and the endothelialization of the stent surface and at the same time the healing of the intervention site. However, the use of the plate-like elements of the very construction of the new inventive intravascular stent, especially for coronary vessels, increases the surface area of interaction of the above-mentioned cells with the inner stent covering, thus significantly improving stent performance.

The primary advantage of the outer coating of the inventive intravascular stent, especially for coronary vessels, is its targeted effect on the cells of the blood vessel wall. The convex structure of the outer coating enters the above wall during stent implantation and provides desired penetration of the wall with a view to gradually releasing the drugs inhibiting cellular proliferation. Furthermore, the central arrangement of the coating and incomplete coverage of the outer surface of the stent construction isolates the action of the cellular proliferation inhibiting compounds, which does not limit the re-endothelialization processes of the stent implantation site. On the other hand, the shaping of the outer stent covering located on the outer surface of the oval plate of the elongated segment in the shape of a “+” symbol or the letter “X” and in the shape of letter “Y” on the outer surface of the plate-like end of the connecting elements to connect the elongated lines of the extreme stent segments significantly reduces the level of drug release and ensures its topical action as well as a proper separation from endothelial cells.

The primary advantage of the new inner covering of the inventive intravascular stent, especially for coronary vessels, is the interaction with the late endothelial progenitor cells and the effect on the vascular endothelial cells. The layer of the inner stent covering localised directly on the stent inner surface captures the late endothelial progenitor cells from the bloodstream and induces endothelialization of the inner surface of the stent construction. The outer layer of the inner stent covering makes it in turn possible to activate or induce the tropomyosin-1 expression in endothelial cells, which are positionally stabilised on the inner layer of the covering, which significantly accelerates the pace of their migration, with the efficient mechanism of cell-cell type bond formation being at the same time maintained. Moreover, endothelial cells with stabilised actin cytoskeleton through the tropomyosin-1 expression do not lose the ability to form intercellular connections in the presence of proinflammatory agents. Hence, the two-layered covering of the inner stent construction significantly accelerates the endothelialization of the stent construction and the re-endothelialization of the stent implantation site, which reduces the possibility of the appearance of restenosis by substantially reducing neointima hyperplasia. The use of the plate-like construction elements of the inventive intravascular stent, especially for coronary vessels, increases in contrast the surface area of interaction of the above-mentioned cells with the inner stent covering, thus improving stent performance.

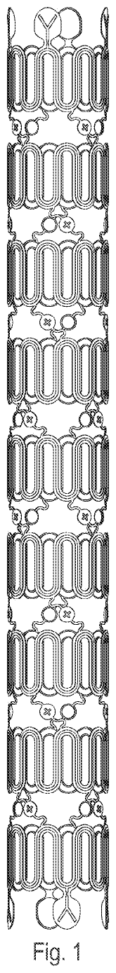

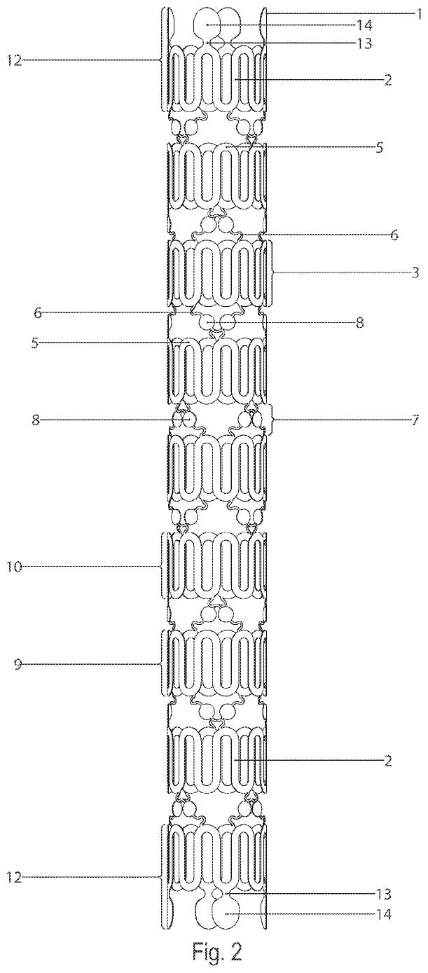

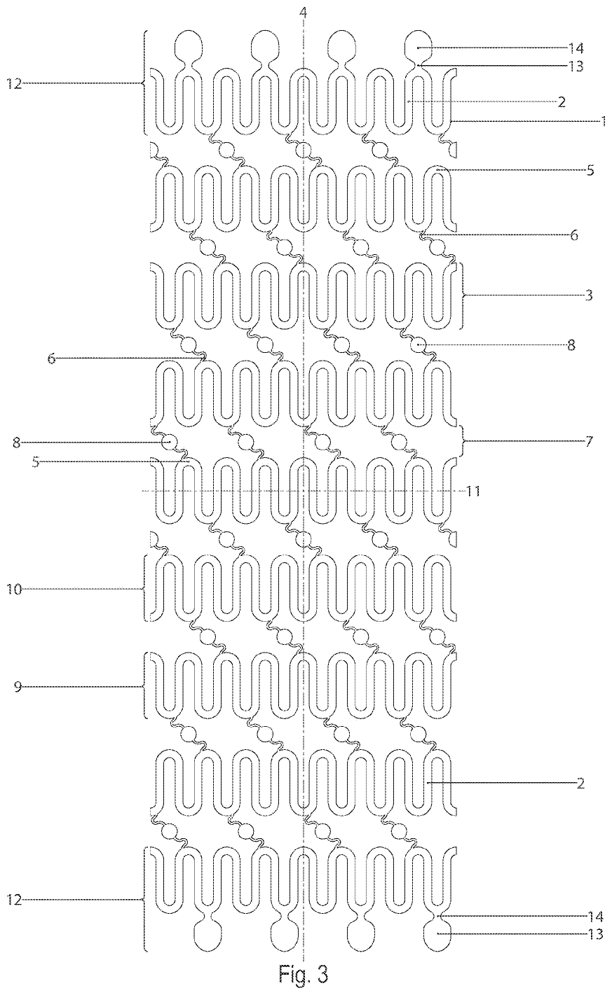

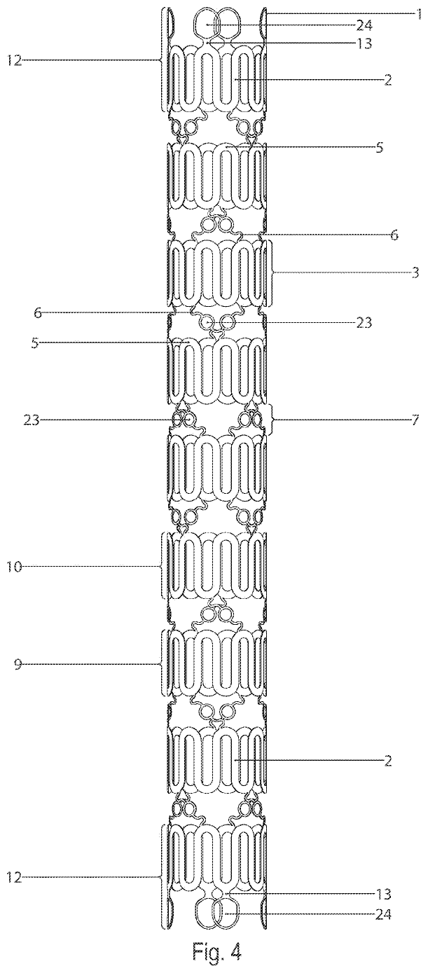

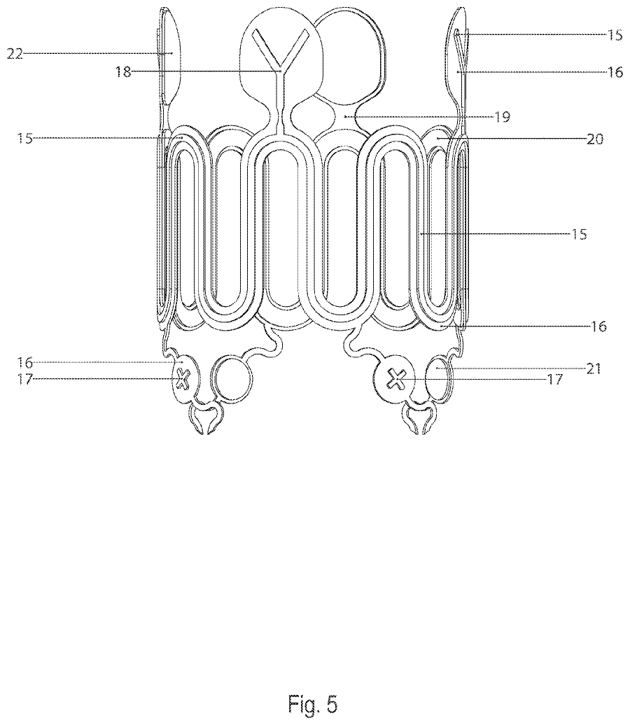

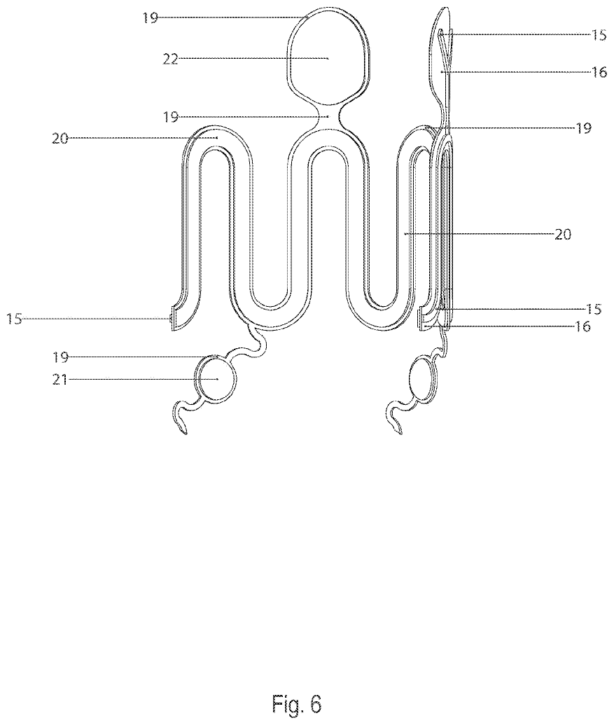

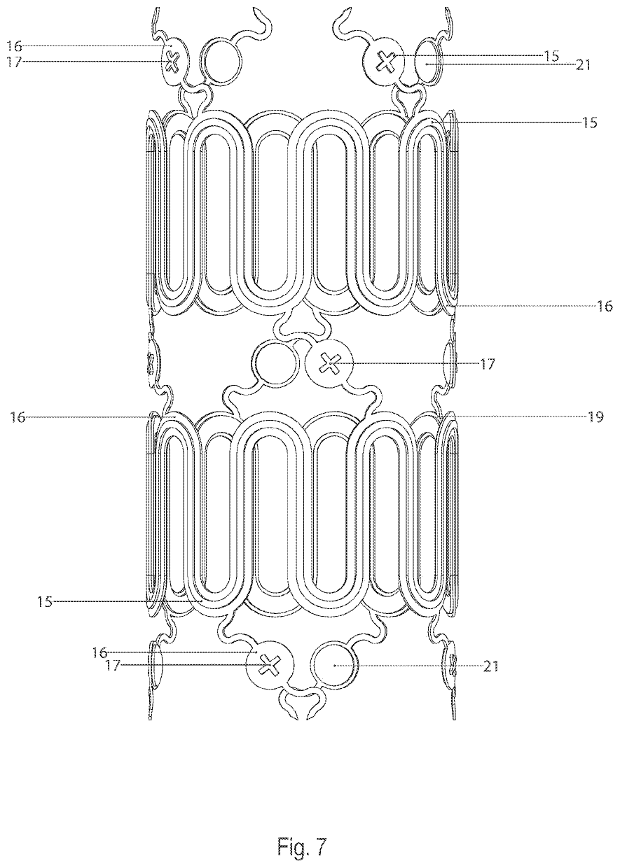

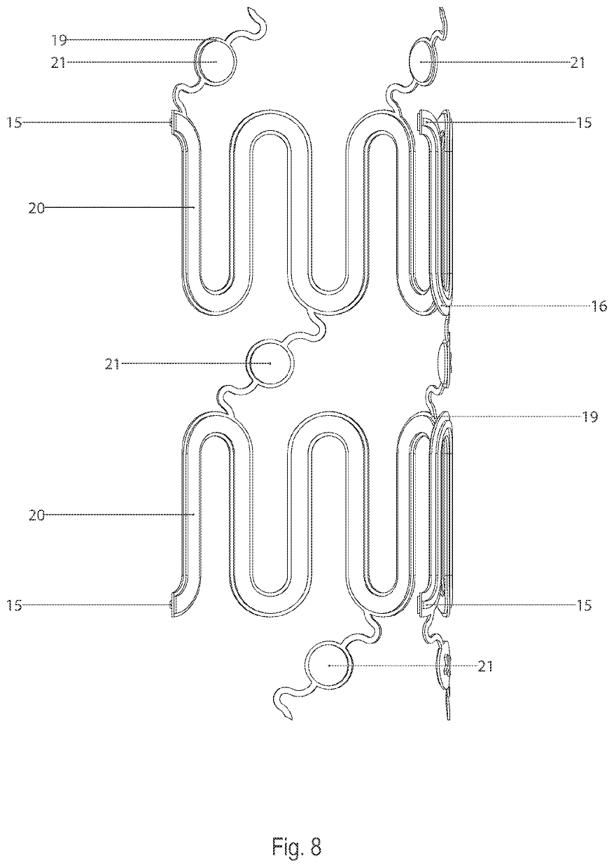

The invention is explained in more detail in the embodiments shown in the drawings, where FIG. 1 shows a general view of the construction of the intravascular stent, especially for coronary vessels, together with the outer and inner stent covering, FIG. 2 shows a skeleton of the construction of the intravascular stent, especially for coronary vessels, FIG. 3 shows a skeleton of the construction of the intravascular stent, especially for coronary vessels, cut longitudinally and flattened, FIG. 4 shows a skeleton of the construction of the intravascular stent, especially for coronary vessels, with rings formed from plate-like elements, FIG. 5 shows the extreme fragment of the internally and externally coated intravascular stent, especially for coronary vessels, the fragment including the plate-like ends of the connecting elements to connect the elongated lines of the extreme main stent segments in the shape of a round-point spade as well as connecting segments with oval plates, FIG. 6 shows the extreme fragment of the internally and externally coated intravascular stent with the inner surface made visible, FIG. 7 shows the middle fragment of the internally and externally coated intravascular stent, especially for coronary vessels, the fragment including mirrored main segments as well as connecting segments with oval plates, and FIG. 8 shows the middle fragment of the internally and externally coated intravascular stent with the inner surface made visible.

### EXAMPLE 1

The skeleton of the intravascular stent is in the form of a tube with cut-outs made on the lateral surface in a continuous material of a tube of AISI 316L austenitic steel. These cut-outs form the stent construction skeleton (1) consisting of alternately arranged main and connecting segments in such a manner that the elongated lines (2) of the main segment (3) are situated along the longitudinal stent axis (4) and are connected via U-shaped connecting elements (5) thus creating around the longitudinal stent axis (4) a geometric pattern resembling a meander of gentle edges, whereas the two curves (6) of the connecting segment (7) are in the shape of a sickle and connect the oval plate (8) of the connecting segment (7) to the connecting elements (5) of the elongated lines (2) of the main segment (3). The advantage of the segmented construction of the stent is its high resistance to external forces, with proper pliability being at the same time maintained. At the same time, the next main segment (9) of the stent is a mirrored reflection of the previous segment (10), while the curves (6) of the connecting segment (7) in the shape of a sickle (6) are a mutual mirrored reflection in relation to the oval plate (8) of the connecting segment (7). The connecting segments (7) are arranged parallel to each other along the transverse stent axis (11) and are attached alternately to the next and every second connecting element (5) of the elongated lines (2) of the next main segment (3) thus forming an alpha helix pattern. The end stent segments are the extreme main segments (12), whose every second outer connecting element (5) to connect the elongated lines (2) terminates in gentle passageways (13) with a plate in the shape of a round-point spade (14).

### EXAMPLE 2

The intravascular stent, especially for coronary vessels, realised as in the first example except that the outer stent surface (16) is coated with everolimus, a drug with inhibitory effect on cellular proliferation. The outer stent covering (15) is applied on the central part of the outer stent plane (16) and includes the elongated lines (2) of the main segment (3) and their connecting elements (5) and takes 30% of the outer surface area (16) of the stent construction element. The outer stent covering (15), comprising everolimus, also includes a central part of the plate-like stent construction elements. In the case of the oval plate (8) of the connecting segment (7) the outer stent covering (15) is applied centrally in the shape of the letter “X” (17), whereas the outer surface (16) of the plate-like end of the connecting elements (5) to connect the elongated lines (2) of the extreme stent main segments (12) in the shape of a round-point spade (14) is covered with a coating shaped in the shape of letter “Y” (18) in such a manner that the base of the letter connects to the outer covering (15) of the extreme main segments (12) of the stent. The convex structure of the outer coating (15), comprising everolimus, enters the vessel wall during stent implantation and provides desired penetration of the wall with a view to gradual drug release. On the other hand, the incomplete and centrally arranged covering (15) of the outer surface (16) of the stent provides a more targeted effect of the drug on the cells forming the blood vessel wall, which reduces its negative impact on the re-endothelialization processes of the stent implantation site.

### EXAMPLE 3

The intravascular stent, especially for coronary vessels, realised as in the first example except that the inner surface (19) of its construction is covered with a bilayer accelerating the endothelialization of the stent construction and the re-endothelialization of the stent implantation site, which reduces the possibility of the appearance of restenosis by substantially reducing neointima hyperplasia. The layer localised directly on the stent inner surface are monoclonal anti-CD144 antibodies being covalently immobilised and the layer includes the entire inner surface (19) of the stent construction. The aim of this layer is to interact with the late endothelial progenitor cells circulating in the blood, induce the endothelial cells localised between the stent construction elements to endothelialization of the stent inner surface and positionally stabilise the cells migrating onto the inner surface (19) of the stent. The above layer is partially covered with covalent or electrostatic complexes of complexes of cell-penetrating peptides together with the plasmid DNA of CRISPR/dCas9 system activating the tropomyosin-1 expression (20). This covering is located in the central part of the stent construction elements, taking 70% of their surface area. This coating is located along the inner surface (19) of the stent and includes the elongated lines (2) of the main segment (3) and their connecting elements (5). It also includes the central part of the inner surface (19) of the oval plate (8) of the connecting segment (7) and is shaped in an oval shape (21). The covering is also applied on the central part of the inner surface (19) of the plate-like end of the connecting elements (5) to connect the elongated lines (2) of the extreme main stent segments (12) in the shape of a round-point spade (14), where it is shaped in the shape of a round-point spade (22). The task of the inner covering layer (20) of the stent, the layer localised from the bloodstream side, is to activate the tropomyosin-1 expression in endothelial cells which are positionally stabilised on the inner layer of the covering. The induction of tropomyosin-1 expression by affecting the actin cytoskeleton of endothelial cells will significantly accelerate the pace of their migration while maintaining the efficiently working mechanism of cell-cell type bond formation. Additionally, the covering of the plate-like elements of the stent construction increases the surface area of interaction of cells with the inner stent covering, thus improving the performance of the stent inner coating.

### EXAMPLE 4

The intravascular stent, especially for coronary vessels, realised as in the first or second or third example, with the stent construction being realised by cut-outs from a tube of nickel-titanium alloy. Then the oval plates (8) of the connecting segment (7) of the stent or the plate-like ends of the connecting elements (5) to connect the elongated lines (2) of the extreme segments (12) of the stent in the shape of a round-point spade (14) are oval rings (23) made by cuts in the continuous material or rings (24) in the shape of a round-point spade, in which platinum markers of a reduced transparency for X-rays are placed, which improves the quality of the stent implantation procedure and makes it possible to directly monitor how the stent is arranged with respect to the vessel wall and whether the stent is properly fitted in the stenosis site.

### EXAMPLE 5

The intravascular stent, especially for coronary vessels, realised as in the first or the fourth example, with the outer surface (16) of the stent construction being covered with everolimus, as in the second example, and with the inner surface (19) of the stent construction being covered, as in the third example, with two layers of a biodegradable and biocompatible polymer comprising monoclonal anti-CD144 antibodies and covalent or electrostatic complexes of cell-penetrating peptides together with the plasmid DNA of CRISPR/dCas9 system activating the tropomyosin-1 expression. The outer stent coating in a direct and targeted manner affects the cells forming the blood vessel wall by inhibiting their proliferation. On the other hand, the central arrangement of the coating and incomplete coverage of the outer surface of the stent construction isolates the action of the drug, which does not limit the re-endothelialization processes of the stent implantation site. The inner stent covering, in turn, in a coordinated manner affects the late endothelial progenitor cells and the endothelial cells of the vessels in order to accelerate the pace of the endothelialization of the stent construction and the re-endothelialization of the stent implantation site, which reduces the possibility of the appearance of restenosis by substantially reducing neointima hyperplasia. On the other hand, the covering of the plate-like elements of the stent construction increases the surface area of interaction of the above-mentioned cells, thus improving stent performance.

1.-17.(canceled)

18. An intravascular stent comprising:
a main body in having a form of a tube with a lateral surface and an inner surface, which has a repeated, symmetrical segmented pattern made by cut-outs on the lateral surface of the tube of a continuous material covered with biodegradable and biocompatible polymer comprising substances affecting cells, where the cut-outs forming the segmented patterns of the stent construction at same time form elongated lines of a main segment situated around a longitudinal stent axis and are connected via U-shaped connecting elements thus creating around the longitudinal axis of the stent a geometric pattern resembling a meander of gentle edges, and two curves in having shape of letter “V” with rounded edges together with an oval plate form a connecting segment to connect with connectors of the elongated lines of the main segment, wherein every next main segment is a mirror reflection of a corresponding previous segment.

19. The intravascular stent of claim 18, wherein V-shaped curves of the connecting segment are a mutual mirror reflection in relation to the oval plate of the connecting segment.

20. The intravascular stent of claim 18, wherein the connecting segments are arranged parallel to each other along a transverse stent axis and are attached alternately to next connector of the elongated lines of the next main segment thus forming an alpha helix pattern.

21. The intravascular stent of claim 18, wherein the connecting segments of the stent are attached to every second connecting element of the elongated lines of the main segment.

22. The intravascular stent of claim 18, wherein some stent segments are extreme main segments, whose every second outer connecting element to connect the elongated lines terminates in gentle passageways with a plate in the shape of a round-point spade.

23. The intravascular stent of claim 22, wherein the oval plates of the connecting segment of the stent or a plate-like ends of the connecting elements to connect the elongated lines of the extreme main segments of the stent in the shape of a round-point spade are oval rings or rings in the shape of a round-point spade to place markers of a reduced transparency for X-rays, especially of platinum or tantalum or gold.

24. The intravascular stent of claim 23, wherein an outer covering of the stent comprises drugs, and in particular the drugs inhibiting cellular proliferation, and forms a layer covering an outer surface of the stent.

25. The intravascular stent of claim 24, wherein the outer covering of the stent takes up to 50%, but not less than 10%, of the outer surface of the stent.

26. The intravascular stent of claim 24, wherein the outer covering of the stent is located centrally, along the outer surface of the stent and includes the elongated lines of the main segment and their connecting elements.

27. The intravascular stent of claim 24, wherein the outer covering of the stent is located centrally on the outer surface of the oval plate or a marker-filled ring of the connecting segment and is shaped in the shape of a “+” symbol or the letter “X”.

28. The intravascular stent of claim 24, wherein the outer covering of the stent is located centrally on the outer surface of the plate-like end or a marker-filled ring of the connecting elements to connect the elongated lines of the extreme main stent segments in the shape of a round-point spade and is shaped in the shape of the letter “Y” n such a manner that the base of the letter connects to the outer covering of the extreme main segments of the stent.

29. The intravascular stent of claim 18, wherein entire inner surface of the stent, including the inner surface of a marker-filled oval ring of the connecting segment of the stent and the inner surface of the marker-filled ring of the connecting elements to connect the elongated lines of the extreme main stent segments in the shape of a round-point spade, is covalently immobilized or in a film with monoclonal anti-CD144 antibodies.

30. The intra vascular stent of claim 18, wherein an inner covering of the stent comprises a system of induction of tropomyosin-1 expression, especially covalent or electrostatic complexes of cell-penetrating peptides together with CRISPR/dCas9 system activating the tropomyosin-1 expression or with expression vectors determining the expression of human recombinant tropomyosin-1, or with stabilized mRNA molecules coding human tropomyosin-1, and forms the layer covering the inner surface of the stent main body.

31. The intravascular stent of claim 30, wherein the inner covering of the stent takes up to 90%, but not less than 50%, of the inner surface of the stent construction element.

32. The intravascular stent of claim 30, wherein the inner covering of the stent is located centrally, along the inner surface of the stent and includes the elongated lines of the main segment and their connectors.

33. The intravascular stent of claim 30, wherein the inner covering of the stent is located centrally on the inner surface of the oval plate or the marker-filled ring of the connecting segment and is shaped in an oval shape.

34. The intravascular stent of claim 30, wherein the inner covering of the stent is located centrally on the inner surface of the plate-like end or a marker-filled ring of the connecting elements to connect the elongated lines of the extreme main segments of the stent and is shaped in the shape of a round-point spade.
